(19) United States
(12) Patent Application Publication
(10) Pub. No.: US 2011/0230775 A1
(43) Pub. Date: Sep. 22, 2011

(54) IMAGING APPARATUS FOR IMAGING A HEART

(75) Inventors: Maya Ella Barley, Eindhoven (NL); Joachim Kahlert, Aachen (DE)

(73) Assignee: KONINKLIJKE PHILIPS ELECTRONICS N.V., EINDHOVEN (NL)

(21) Appl. No.: 13/130,395

(22) PCT Filed: Nov. 20, 2009
(86) PCT No.: PCT/IB09/55221

§ 371 Date: May 20, 2011

(30) Foreign Application Priority Data
Nov. 24, 2008 (EP) 08169736.9

(51) Int. Cl. A61B 5/02

(52) U.S. Cl. 600/508

(57) ABSTRACT

An imaging apparatus for imaging a heart is provided, wherein the imaging of the heart is improved such that conclusions about regions of the heart having an abnormal behaviour can be made more accurate and more optimal. The imaging apparatus comprises a first site determination unit for determining a first site of the heart comprising a first property type like a fractionated electrogram (70,71,74,75) and a second site determination unit for determining a second site comprising a second property type like a ganglionated plexus (72,73). The first site and the second site are causally related and displayed on a display unit. Since the displayed first and second sites are causally related to each other, a further information is given, i.e. the causal relation, which assists a user in finding regions of the heart showing an abnormal behaviour.

### FIELD OF THE INVENTION

The present invention relates to an imaging apparatus, an imaging method and an imaging computer program for imaging a heart.

### BACKGROUND OF THE INVENTION

The article “Integration of Three-Dimensional Scar Maps for Ventricular Tachycardia Ablation With Positron Emission Tomography-Computed Tomography”, T. Dickfeld et al., Journal of the American College of Cardiology Foundation, Cardiovascular Imaging, 1:73-82, 2008 describes a system for determining sites of scar tissue of a heart and for co-displaying these sites with an electroanatomical map of the heart.

The system has the drawback that a tremendous volume of electroanatomical data is presented that, for example, an electrophysiologist must mentally parse and interpret in order to determine, for example, optimal ablation sites. This mental process is time-consuming and often difficult and may lead to inaccurate or sub-optimal conclusions, in particular, on optimal ablation sites.

### SUMMARY OF THE INVENTION

It is an object of the present invention to provide an imaging apparatus, an imaging method and an imaging computer program for imaging a heart, wherein the imaging of the heart is improved such that conclusions about regions of the heart having an abnormal behavior can be made more accurate and more optimal.

In an aspect of the present invention an imaging apparatus for imaging a heart is presented, wherein the imaging apparatus comprises:
a property type providing unit for providing property types of the heart at different locations of the heart,
a first site determination unit for determining a first site of the heart, wherein the first site comprises a first property type of the provided property types,
a second site determination unit for determining a second site of the heart, wherein the second site comprises a second property type of the provided property types and wherein the second site has a causal relation to the first site,
a display unit for displaying the first site and the second site.

Since the first site and the second site are displayed, which are causally related to each other, a user like an electrophysiologist or a radiologist does not only obtain information about the location of the first site and of the second site, but also the information that the first site and the second site are causally related. This further information assists the user in finding regions of the heart showing an abnormal behavior, which could be regarded as an ablation site. Conclusions about an abnormal behavior of a region of the heart can therefore be made more accurate and more optimal.

The first site and the second site are preferentially causally related if the property type of at least one of the first site and the second site causes or promotes the property type of the other of the first site and the second site. It is further preferred that the term “causal relation” relates to the pathophysiological relationship between the first property type of the first site and the second property type of the second site. In particular, the first site and the second site are causally related, if one of the first site and the second site comprises an anatomical property type that could also be regarded as an anatomical feature—which may be found in the healthy human heart (such as a ganglionated plexus) or may be disease-created (such as an area of myocardial infarct)—and if the other of the first site and the second site comprises an electrical property type that could also be regarded as electrical behavior, which is caused or promoted by the anatomical property type (for example ectopic foci or fractionated electrograms that are the electrical triggers or substrate of cardiac arrhythmia).

The first property type and/or the second property type are preferentially property types related to the functioning of the heart. It is further preferred that the first site and the second site comprise tissue of the heart having the first property type and the second property type, respectively. A property type can also be regarded as a property class, wherein one or several properties at a location of the heart are classified in accordance with a predefined classification criterion and wherein the property class of the one or several properties at this location is the property type at this location.

In a preferred embodiment, the first site determination unit comprises a selection unit for allowing a user to select a first property type of the provided property types of the heart, wherein the first site determination unit is adapted to determine the first site of the heart which comprises the selected first property type.

It is also preferred that the imaging apparatus comprises a heart model providing unit for providing a model of the heart, wherein the display unit is adapted to display the first site and the second site on the provided heart model.

It should be noted that the invention is not limited to one first site and one second site only. The first site determination unit can be adapted to determine several first sites and the second site determination unit can be adapted to determine several second sites. Furthermore, the imaging apparatus can also comprise a third site determination unit for determining third sites comprising a third property type, a fourth site determination unit for determining a fourth site comprising a fourth property type et cetera.

Preferentially, the display unit is adapted to display only sites of the heart, which are causally related.

The property type providing unit preferentially comprises an electrogram providing unit for providing an electroanatomical map, which shows electrograms at different locations at a surface of the heart. Furthermore, the property type providing unit can comprise a heart image providing unit for providing an image of the heart like a magnetic resonance, an x-ray computed tomography, a nuclear or a three-dimensional atrioangiography image.

The electrogram providing unit can be an electrogram storing unit, in which an electroanatomical map is stored, or an electrogram measuring unit for measuring an electrogram at different locations at a surface of the heart. The electrogram measuring unit can comprise a contact electrode on a catheter tip for locally stimulating the tissue of the heart, wherein after or during stimulation the electrograms are measured.

The heart image providing unit can be a heart image storing unit in which a heart image is stored or a heart image generation unit for generating an image of the heart. The heart image generation unit is preferentially an imaging modality like a magnetic resonance, an x-ray computed tomography, a nuclear imaging or a three dimensional atrioangiography modality for imaging the heart.

It is further preferred that the property type providing unit is adapted to provide at least one of an anatomical property type and an electrical property type of the heart. In a preferred embodiment, the property type providing unit is adapted to provide at least one of a complex fractionated atrial electrogram, a ganglionated plexus, a re-entrant circuit, scar tissue, a rotor, a pulmonary vein ostium, a slow conduction and fibrosis as a property type of the heart. The property type providing unit can also be adapted to provide an ectopic focus or a mitral valve annulus as property type of the heart. These property types can easily be determined from an electroanatomical map and/or an image of the heart and these property types have a diagnostic value leading, for example, an electrophysiologist to sites of the heart, which have to be ablated. The re-entrant circuits can also be named re-entrant circuit pathways.

In an embodiment, the property type providing unit comprises a property type determination unit for determining the property types of the heart at different locations of the heart based on an electroanatomical map provided by the electrogram providing unit and/or an image of the heart provided by the heart image generation unit. The property type determination unit is preferentially adapted to determine a complex fractionated atrial electrogram, an ectopic focus, a rotor, a high frequency electrogram, a re-entrant circuit or a slow conduction as a property type and their corresponding locations at the heart by using the electroanatomical map and/or the image of the heart. In addition or alternatively, the property type determination unit can be adapted to determine a ganglionated plexus and/or scar tissue, a pulmonary vein ostium and a mitral valve annulus as property type and their location at the heart by using the image of the heart, in particular, by using a magnetic resonance or a x-ray computed tomography image, provided by the heart image providing unit and/or by using the electroanatomical map provided by the electrogram providing unit. The property type determination unit can also be adapted to determine a ganglionated plexus and/or scar tissue and/or a re-entrant circuit based on measuring changes in electrograms following local stimulation. In particular, a re-entrant circuit can be based on entrainment mapping.

The determination of the previously mentioned property types based on an electroanatomical map and/or an image of the heart is known to the person skilled in the art. For some property types this determination will exemplarily be explained in the following.

For determining the property type ganglionated plexus preferentially an area within the borders of a ganglionated plexus is identified by sequentially applying at multiple locations high frequency local stimulation (for example 0.1 V, 5 Hz square waves of duration 2 ms) for several seconds while observing the electrogram for a vagal response (i.e. a prolongation of the R-R interval). This stimulation process is repeated until the borders of the ganglionated plexus have been fully mapped. This determination of a ganglionated plexus is described in more detail in the article “How to perform ablation of the parasympathetic ganglia of the left atrium”, Lemery et al., Heart Rhythm, 2006. 3 (10): p. 1237-1239, which is herewith incorporated by reference.

The property type scar tissue is preferentially determined by subthreshold stimulation of the endocardium. The resulting local electrograms are measured a few millimeters from a pacing electrode. Scar regions are characterized by low-voltage (preferentially smaller than 1.5 mV) multiphasic electrograms. A more detailed description of this determination of the property type scar tissue is described in more detail in the article “Electrically unexcitable scar mapping based on pacing threshold for identification of the reentry circuit isthmus: feasibility for guiding ventricular tachycardia ablation”, Soejima, K. et al., Circulation, 2002. 106 (13): p. 1678-83, which is herewith incorporated by reference.

To determine the property type re-entrant circuit, in particular, to determine the pathways of re-entrant circuits, suprathreshold pacing to mimic the ventricular tachycardia (pace mapping) is performed at locations in or near scar tissue. This technique is based on the principle that pacing within the re-entrant circuit will result in an identical surface electrocardiogram morphology to that of the clinical ventricular tachycardia. A more detailed description of the determination of the pathways of re-entrant circuits is described in more detail in the article “Mapping for ventricular tachycardia”, Dixit, S. and D. J. Callans, Card Electrophysiol Rev, 2002. 6 (4): p. 436-41, which is therewith incorporated by reference.

Entrainment mapping is a gold-standard for guidance of a catheter to an optimal site for ablation. Entrainment mapping is performed after the re-entrant circuit site has been localized, and is used to identify the optimal site for ablation. It ascertains whether the current location of the ablation catheter tip is within the re-entrant circuit by comparing the ventricular tachycardia cycle length with the post-pacing interval (the period between administration of a pacing stimulus and return of the stimulus to the pacing site). If they are equal, the position of the ablation catheter tip is within the re-entrant circuit. This entrainment mapping is described in more detail in “Catheter ablation of monomorphic ventricular tachycardia”, Stevenson, W. G., Curr Opin Cardiol, 2005. 20 (1): p. 42-7, which is herewith incorporated by reference.

In a further embodiment the property type providing unit is a storing unit, in which the property types and their locations at the heart are stored already. The property type providing unit can also be a data receiving unit for receiving data indicating at which locations of the heart which property types are present and for providing the received data to the first site determination unit and the second site determination unit.

It is further preferred that the second site determination unit comprises a causality determination unit for determining among the provided property types of the heart a property type that has a causal relation to the first property type, wherein this determined property type is the second property type and wherein the second site determination unit is adapted to determine the second site as the site where the determined second property type is located. It is also preferred that the causality determination unit comprises a storing unit for storing causal property type groups, wherein property types of a causal property type group comprise a causal relation and wherein the causality determination unit is adapted to determine that the first property type and a further property type among the provided property types are causally related, if the first property type and the further property type belong to the same causal property type group. The further property type belonging to the same causal property type group is preferentially the second property type. This allows to fast and accurately determine property types, which are causally related, by looking in the storing unit whether two property types belong to the same causal property type group. Furthermore, further causal relations between property types can easily be introduced into the imaging apparatus by adding new causal property type groups to the storing unit.

In a preferred embodiment, at least one of the following causal property type groups is stored in the storing unit:
complex fractionated atrial electrogram and ganglionated plexus,
re-entrant circuit and scar tissue,
rotor and pulmonary vein ostium,
ectopic focus and pulmonary vein ostium,
slow conduction and fibrosis,
slow conduction and ischemia.

These causal property type groups have a causal relation, and displaying a first site and a second site, wherein the corresponding first property type and the corresponding second property type belong to one of these causal property type groups, can lead an electrophysiologist to a site of the heart, which has to be ablated.

It is further preferred that the imaging apparatus further comprises a causality level determination unit for determining a level of causality between the first site and the second site. The level of causality gives a user a further indication with respect to an abnormal behavior of a region of the heart. In particular, if the level of causality is higher, at least one of the first site and the second site is more likely a site, which has to be ablated.

In an embodiment, the causality level determination unit is adapted to determine the level of causality between each of several first sites and a second site being the only second site or being a selected second site out of several second sites. Furthermore, the causality level determination unit can be adapted to determine the level of causality between each of several second sites and a first site being the only first site or being a selected first site out of several first sites. The causality level determination unit comprises preferentially a selection unit for selecting a first site and/or a second site, for example, a graphical user interface.

In a preferred embodiment, the causality level determination unit is adapted to determine the level of causality based on the distance between the first site and the second site.

It is further preferred that a smaller distance between the first site and the second site corresponds to a higher level of causality, in particular, if the first site comprises a re-entrant circuit and the second site comprises scar tissue or vice versa.

It is further preferred that the causality level determination unit is adapted to determine the level of causality based on the density of one of the first site and the second site within a predefined area around the other of the first site and the second site. The first property type of the first site can alter the electrical substrate of an area of tissue and may be expected to do this comprehensively in the first site and in the predefined area around the first site. If the density of second sites comprising the second property type that is causally related to the first property type within this predefined area is higher, it is assumed that the level of causality between the first site and the second sites is increased. For example, a ganglionated plexus as a first property type in the first site can alter the electrical substrate of an area of tissue (for instance, by autonomic nervous input), and may be expected to do this comprehensively within this area of tissue, which could be regarded as the predefined area. That is, the density of second sites with the second property type (e.g. complex fractionated atrial electrogram) within the predefined area indicates a higher level of causality with the first site which comprises, in this example, a ganglionated plexus. In an embodiment, the predefined area is defined based on the provided property types, in particular, based on at least one of the first property type and/or the second property, and their locations in the heart. For example, if the first property type is a ganglionated plexus an alteration of the electrical substrate of an area of tissue is determined, for example, based on an electroanatomical map, wherein the predefined area is predefined by defining an area in which the electrical substrate has been altered. The predefined area can also be predefined by a user like an electrophysiologist.

It is further preferred that the causality level determination unit is adapted to determine the level of causality based on the location, which is preferentially an anatomical location, of at least one of the first site and the second site. In particular, a first site comprising a complex fractionated atrial electrogram as first property type may be a single first site or several first sites may be present comprising complex fractionated atrial electrograms, which cluster into groups in known anatomical regions. Furthermore, each ganglionated plexus is known to provide autonomic nervous input to one or more particular areas of heart tissue as it is, for example, disclosed in the article “Autonomic Mechanism to Explain Complex Fractionated Atrial Electrograms (CFAE)”, Lin et al., J. Cardiac Electrophysiol, 2007. 18 (11): p. 1197-1205. Therefore, if the second property type of the second site is a ganglionated plexus, the level of causality between the first site comprising the first property type being a complex fractionated atrial electrogram and a second site having the second property type being a ganglionated plexus is larger, if the first site and the second site are located around the left inferior pulmonary vein ostium and inferior. The level of causality is smaller if the first site and second site are located around the right superior pulmonary vein ostium and inferior to the left-inferior pulmonary vein, respectively

It is further preferred that the display unit is adapted to display the first site and/or the second site depending on the determined level of causality. Thus, the display unit does not only display the first site and the second site, which are causally related, but also the level of causality. For example, the color of the first site and/or the second site can be adapted to the level of causality or the intensity or brightness of the displayed first site and second site can depend on the respective level of causality. If several first sites and/or second sites are present, the different first sites and/or second sites can be displayed differently in dependence on their level of causality, i.e. different first sites and/or second sites can comprise different level of causalities. For example, all first sites can be displayed in a first color and all second sites can be displayed in a second color, wherein the color intensity or brightness depends on the level of causality, for example, if the level of causality is larger, the intensity or brightness can be larger. This further improves, for example, the guidance of an electrophysiologist to sites, which should be ablated.

In a further aspect of the present invention an energy application apparatus for applying energy to a heart is presented, wherein the energy application apparatus comprises an energy application unit for applying energy to the heart and an imaging apparatus as defined in claim 1.

In a further aspect of the present invention an imaging method for imaging a heart is presented, wherein the imaging method comprises following steps:
providing property types of the heart at different locations of the heart,
determining a first site of the heart, wherein the first site comprises a first property type of the provided property types,
determining a second site of the heart, wherein the second site comprises a second property type of the provided property types and wherein the second site has a causal relation to the first site,
displaying the first site and the second site.

In a further aspect of the present invention a computer program for imaging a heart is presented, wherein the computer program comprises program code means for causing an imaging apparatus as defined in claim 1 to carry out the steps of the imaging method as defined in claim 13, when the computer program is run on a computer controlling the imaging apparatus.

It shall be understood that the imaging apparatus of claim 1, the energy application apparatus of claim 12, the imaging method of claim 13, and the computer program of claim 14 have similar and/or identical preferred embodiments as defined in the dependent claims.

It shall be understood that a preferred embodiment of the invention can also be any combination of the dependent claims with the respective independent claim.

### BRIEF DESCRIPTION OF THE DRAWINGS

These and other aspects of the invention will be apparent from and elucidated with reference to the embodiments described hereinafter. In the following drawings

FIG. 1 shows schematically and exemplarily a representation of an embodiment of an imaging apparatus for imaging a heart in accordance with the invention,

FIG. 2 shows exemplarily a flowchart illustrating an embodiment of an imaging method for imaging a heart in accordance with the invention,

FIG. 3 shows schematically and exemplarily a representation of an embodiment of an energy application apparatus for applying energy to a heart in accordance with the invention,

FIG. 4 shows schematically and exemplarily electrodes on a holding structure of the embodiment of the imaging apparatus in an unfolded condition,

FIG. 5 shows schematically and exemplarily the electrodes with the holding structure in a folded condition,

FIG. 6 shows schematically and exemplarily a control unit of the embodiment of the energy application apparatus,

FIG. 7 shows determined first and seconds sites on a model of the heart and

FIG. 8 shows exemplarily a flowchart illustrating an embodiment of an imaging method for imaging a heart in accordance with the invention.

### DETAILED DESCRIPTION OF EMBODIMENTS

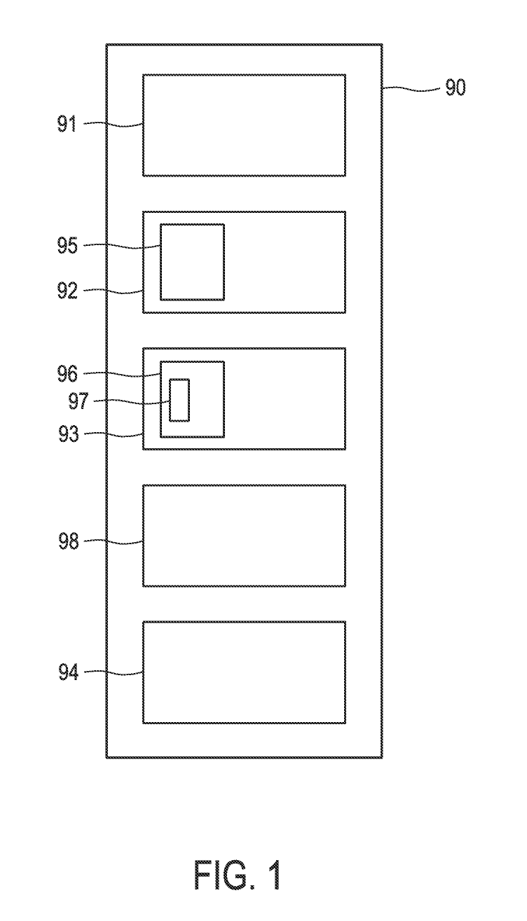

FIG. 1 shows schematically and exemplarily an embodiment 90 of an imaging apparatus for imaging a heart. The imaging apparatus comprises a property type providing unit 91 for providing property types of the heart at different locations of the heart, a first site determination unit 92 for determining a first site of the heart, wherein the first site comprises a first property type of the provided property types, and a second site determination unit 93 for determining a second site of the heart, wherein the second site comprises a second property type of the provided property types and wherein the second site has a causal relation to the first site. The imaging apparatus 90 further comprises a display unit 94 for displaying the first site and the second site.

The first site and the second site are causally related if the property type of at least one of the first site and the second site causes or promotes the property type of the other of the first site and the second site. The first property type and the second property type are property types related to the functioning of the heart, and the first site and the second site comprise tissue of the heart having the first property type and the second property type, respectively.

In this embodiment, the first site determination unit 92 comprises a selection unit 95 for allowing a user to select a first property type of the provided property types of the heart, wherein the first site determination unit 92 is adapted to determine the first site of the heart which comprises the selected first property type.

Furthermore, in this embodiment the property type providing unit 91 is a storing unit, in which the property types and their locations on the heart are stored already. For example, a model of the heart can be stored in the storing unit, wherein property types are assigned to locations on the model. In another embodiment, the property type providing unit can also be a data receiving unit for receiving data indicating at which locations of the heart which property types are present and for providing the received data to the first site determination unit and the second site determination unit, or the property type providing unit can be adapted to receive an electroanatomical map and/or a model of the heart and comprises a property type determining unit for determining the property type and their locations based on the electroanatomical map and/or the model of the heart.

In a further embodiment, the property type providing unit can comprise an electrogram providing unit for providing an electroanatomical map, which shows electrograms at different locations at a surface of the heart. Furthermore, the property type providing unit can comprise a heart image providing unit for providing an image of the heart like a magnet resonance, an x-ray computed tomography, a nuclear or a three-dimensional atrioangiography image.

The electrogram providing unit can be an electrogram storing unit, in which an electroanatomical map is stored, or an electrogram measuring unit for measuring an electrogram at different locations at a surface of the heart. The electrogram measuring unit can comprise a contact electrode on a catheter tip for locally stimulating the tissue of the heart, wherein after or during stimulation the electrograms are measured. The heart image providing unit can be a heart image storing unit in which a heart image is stored or a heart image generation unit for generating an image of the heart. The heart image generation unit is preferentially an imaging modality like a magnetic resonance, an x-ray computed tomography, a nuclear imaging or a three dimensional atrioangiography modality for imaging the heart.

In this embodiment, the property type providing unit 91 is adapted to provide at least one of an anatomical property type and an electrical property type of the heart. In particular, the property type providing unit 91 is adapted to provide at least one of a complex fractionated atrial electrogram, a ganglionated plexus, a re-entrant circuit, scar tissue, a rotor, a pulmonary vein ostium, a slow conduction, fibrosis, an ectopic focus and a mitral valve annulus as a property type of the heart.

The second site determination unit 93 comprises a causality determination unit 96 for determining among the provided property types of the heart a property type that has a causal relation to the first property type, wherein this determined property type is the second property type and wherein the second site determination unit 93 is adapted to determine the second site as the site where the determined second property type is located. The causality determination unit 96 comprises a storing unit 97 for storing causal property type groups, wherein property types of a causal property type group comprise a causal relation and wherein the causality determination unit 96 is adapted to determine that the first property type and a further property type among the provided property types are causally related, if the first property type and the further property type belong to the same causal property type group. The further property type belonging to the same causal property type group is the second property type. In the storing unit 97 at least one of the following causal property type groups is stored:
complex fractionated atrial electrogram and ganglionated plexus,
re-entrant circuit and scar tissue,
rotor and pulmonary vein ostium,
ectopic focus and pulmonary vein ostium,
slow conduction and fibrosis,
slow conduction and ischemia.

The imaging apparatus 90 further comprises a causality level determination unit 98 for determining a level of causality between the first site and the second site. The level of causality gives a user a further indication with respect to an abnormal behavior of a region of the heart. In particular, if the level of causality is higher, at least one of the first site and the second site is more likely a site, which has to be ablated.

The causality level determination unit 98 is adapted to determine the level of causality based on at least one of the following criteria: a) the distance between the first site and the second site, b) the density of one of the first site and the second site within a predefined area around the other of the first site and the second site, and c) the location, which is preferentially an anatomical location, of at least one of the first site and the second site.

The display unit 94 is preferentially adapted to display the first site and/or the second site depending on the determined level of causality. Thus, preferentially the display unit 94 does not only display the first site and the second site, which are causally related, but also the level of causality. For example, the color of the first site and/or the second site can be adapted to the level of causality or the intensity or brightness of the displayed first site and second site can depend on the respective level of causality. If several first sites and/or second sites are present, the different first sites and/or second sites can be displayed differently in dependence on their level of causality, i.e. different first sites and/or second sites can comprise different level of causalities. For example, all first sites can be displayed in a first color and all second sites can be displayed in a second color, wherein the color intensity or brightness depends on the level of causality, for example, if the level of causality is larger, the intensity or brightness can be larger.

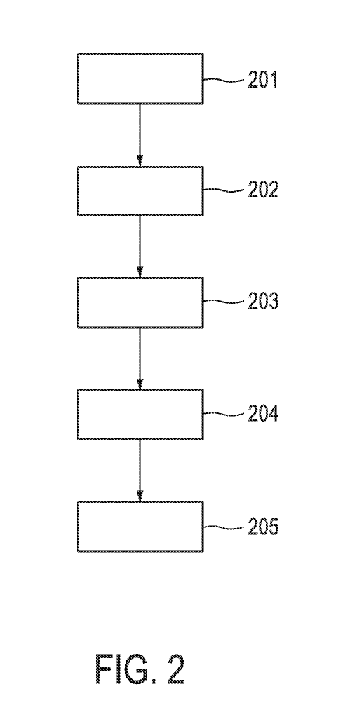

In the following an embodiment of an imaging method for imaging the heart by using the imaging apparatus 90 will be exemplarily described with reference to a flowchart shown in FIG. 2.

In step 201 the property type providing unit 91 provides property types of the heart at different locations of the heart, and in step 202 the first site determination unit 92 determines a first site of the heart, wherein the first site comprises a first property type of the provided property types. Preferentially, a user selects a first property type of the provided property types of the heart by using the selection unit 93 and the first site determination unit 92 determines the first site of the heart which comprises the selected first property type.

In step 203 the second site determination unit 93 determines a second site of the heart, wherein the second site comprises a second property type of the provided property types and wherein the second site has a causal relation to the first site. This is preferentially performed by looking in the storing unit 97 for a causal property group comprising the determined first property type and by determining a property type of a causal property group comprising the first property type as the second property type, wherein the location of this second property type is determined as the second site.

In step 204 the causality level determination unit 98 determines a level of causality between the first site and the second site, and in step 205, the first site and the second site are displayed on the display unit 94, preferentially depending on the determined causality level.

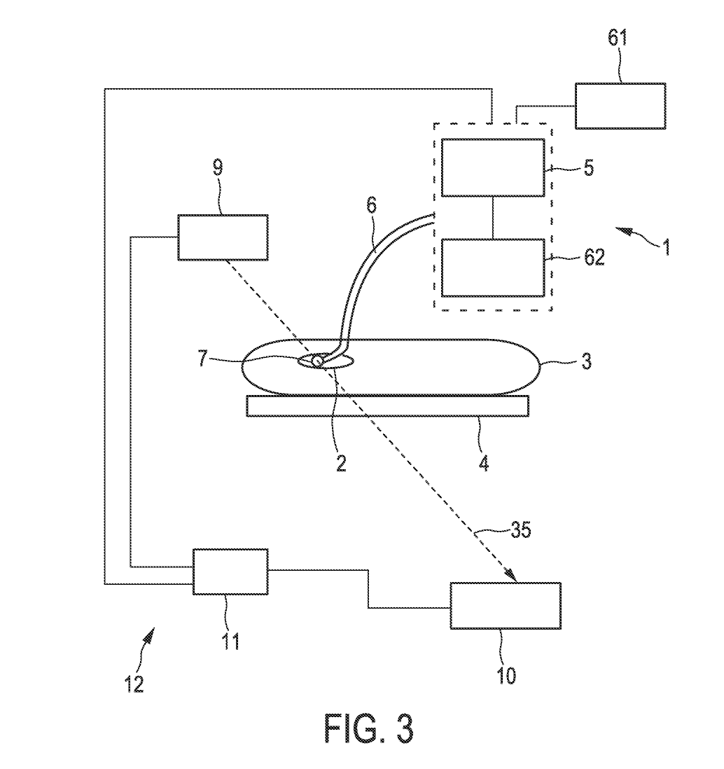

FIG. 3 shows an energy application apparatus 1 for applying energy to a heart 2 comprising an imaging apparatus in accordance with the invention. The energy application apparatus comprises a tube, in this embodiment a catheter 6, and an arrangement 7 of electrodes for measuring electrical signals of the heart 2. The arrangement 7 of electrodes is connected to a control unit 5 via the catheter 6. The catheter 6 with the arrangement of electrodes can be introduced into the heart 2, which is, in this embodiment, a heart 2 of a patient 3 located on a patient table 4, wherein the catheter 6 is steered and navigated to the heart chambers by a steering unit 62 using built-in guiding means (not shown). In another embodiment, the steering unit 62 can comprise an introducer for steering and navigating the catheter 6 to guide the catheter 6 passively into the heart 2. The steering unit 62 can be adapted for steering the arrangement 7 of electrodes manually and/or the steering unit 62 can comprise a robotic system for robotically steering the arrangement 7 of electrodes. This allows steering the arrangement 7 of electrodes to a desired region within the heart, in particular, at an endocardial surface of a heart chamber.

The dashed box in FIG. 3 indicates that both, the control unit 5 and the steering unit 62, are coupled to the catheter 6 comprising the arrangement 7 of electrodes.

During introduction of the arrangement 7 and the catheter 6 into the heart 2 a heart image providing unit 12, which is in this embodiment a fluoroscopy device, generates images of the heart 2 and the arrangement 7. This heart image providing unit 12 preferentially generates images of the heart 2 and the arrangement 7, also if the arrangement 7 is already located within the heart 2.

The heart image providing unit 12, i.e. in this embodiment the fluoroscopy device 12, comprises an x-ray source 9 and a detection unit 10, which are controlled by a fluoroscopy control unit 11. The fluoroscopy device 12 generates x-ray projection images of the heart 2 and of the arrangement 7 in a known way. The x-rays of the x-ray source 9 are schematically indicated by the arrow 35. In another embodiment, instead of a fluoroscopy device, another imaging modality can be used as heart image providing unit for providing a heart image, which, in particular, comprises the heart 2 and the arrangement 7. For example, a magnetic resonance imaging device, an ultrasonic imaging device or a computed tomography imaging device can be used as heart image providing unit for generating and providing an image of the heart 2 and, in particular, of the arrangement 7.

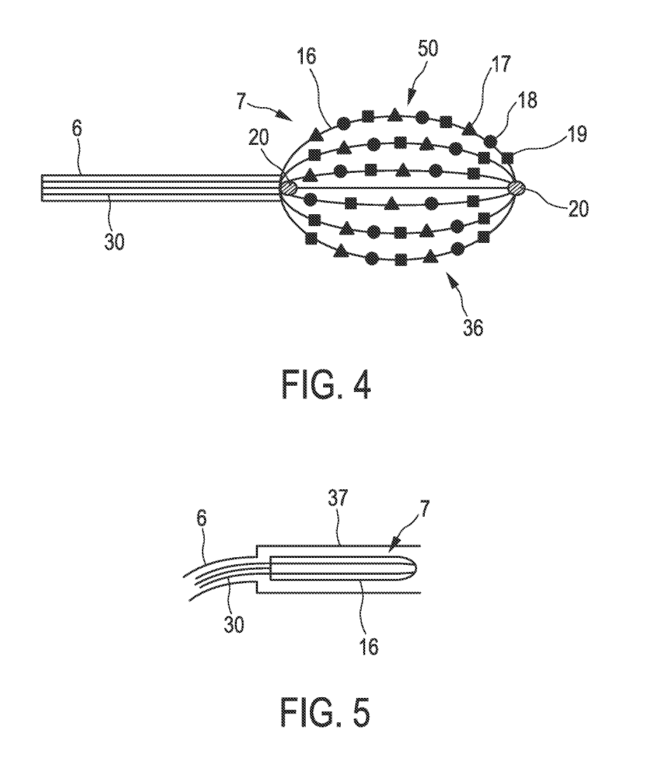

An embodiment of an arrangement 7 of electrodes 17 and a catheter 6 is schematically shown in more detail in FIG. 4. The arrangement 7 is held on a holding structure 50, which is adjustable between a folded condition and an unfolded condition. The holding structure 50 comprises an elongated shape in the folded condition, which is schematically and exemplarily shown in FIG. 5 and which allows to introduce the arrangement 7 into the heart 2. In FIG. 4, the holding structure 50 comprising the electrodes 17 is shown in an unfolded condition.

In this embodiment, the electrodes 17 are used for acquiring electrical signals, which are used for generating an electroanatomical map of the heart. The holding structure further holds temperature sensors 18 for measuring the temperature of the heart and energy emission elements 19 for applying energy to the heart tissue. The temperature sensors 18 can be omitted in another embodiment, i.e. in an embodiment the arrangement 7 only comprises the electrodes 17 and the energy emitting elements 19.

The electrodes 17 are preferentially adapted to measure an electrical signal of the heart 2 like the electrical potential of the heart 2 at different locations. The determined electrical potentials form preferentially electrograms, wherein, since several electrical potentials are determined at different locations of a heart, a map of electrograms can be determined, i.e. an electroanatomical map can be determined.

In an embodiment, the electrodes 17 are adapted to apply energy and to receive energy. This allows sensing the heart by receiving electrical energy for determining an electrical potential, and treating the heart by applying energy using the same electrode, wherein the size of the arrangement of electrodes and of the catheter can be reduced and the influence of the application of energy can easily be monitored at the location, in which the energy has been applied. Especially in this case, the temperature sensors 18 and/or the energy emission elements 19 can be omitted. Furthermore, this allows sensing and stimulating like in pacing catheters. This is especially useful if an electrophysiologist wishes to locate a position within a re-entrant circuit or if the electrophysiologist wishes to delineate the borders of an underlying ganglionated plexus, which can be done by pacing the cardiac tissue and measuring the local change in the R-R interval.

The holding structure 50 has in the unfolded condition preferentially an ellipsoidal or spherical shape, and the electrodes 17 are arranged on the holding structure 50 such that the electrodes 17 are located on the outer surface 36 of the holding structure 50, if the holding structure 50 is in an unfolded condition.

The holding structure 50 comprises a basket made of several splines 16, which comprise the electrodes 17 (indicated by triangles) and, in this embodiment, the energy emission elements 19 (indicated by squares) and the temperature sensors 18 (indicated by circles). The distribution of the electrodes 17, the temperature sensors 18 and the energy emission elements 19 is only schematically and exemplarily in FIG. 4. Preferentially, the electrodes 17 and also possible further temperature sensors 18 and energy emission elements 19 are evenly distributed along these splines 16 and along the outer surface 36.

For acquiring electrical signals from the heart 2 or for applying energy to the heart 2, the outer surface 36 preferentially abuts against a surface of the heart 2 such that the positions of the electrodes 17, the temperature sensors 18 and the energy emission elements 19 remain unchanged relative to the surface of the heart 2 during the acquisition of the electrical signals and during a possible energy application procedure. These fixed positions of the electrodes 17, the temperature sensors 18 and the energy emission elements 19 relative to the heart surface are preferentially achieved by elastic properties of the splines 16 and therefore of the holding structure 50. This elasticity of the splines 16 results in an elastic force, which presses the electrodes 17, the temperature sensors 18 and the energy emission elements 19 against the heart surface. The elasticity of the splines 16 also allows conforming of the outer surface 36 to the heart surface and following a motion of the heart 2, while the electrodes 17, the temperature sensors 18 and the energy emission elements 19 are continuously in contact with the heart surface, or, in other embodiments, the distance between these elements 17, 18, 19 to the heart surface remains continuously constant, even if the heart 2 moves.

The splines 16 comprise preferentially wires made of a memory alloy. In this embodiment, these splines 16 are made of nitinol. For unfolding the arrangement 7, i.e. for unfolding the holding structure 50, the memory effect of the nitinol is used. The nitinol wires are preshaped and elastic as a spring. In the folded condition, which is schematically shown in FIG. 5 and in which the arrangement 7 takes a smaller space, the splines 16 of the arrangements 7 are located within a catheter shaft 37, in particular, in a small pipe within a catheter shaft 37. For unfolding the arrangement 7, i.e. for changing from the folded condition to the unfolded condition, these splines 16 are moved out of the catheter shaft 37, wherein the arrangement 7 forms the outer surface 36, because of the memory effect of the nitinol wires.

FIG. 5 is a schematic view only. In order to enhance the clarity of the folded condition, the illustration shows only some splines 16 of the arrangement 7 and electrodes, temperature sensors and energy emission elements are not shown, although there are preferentially still present.

In other embodiments, other catheters and/or arrangements of one or several electrodes can be used for acquiring electrical signals for generating an electroanatomical map and in particular for applying energy to the heart, and instead of or in addition to using electrodes for applying energy to the heart other energy emitting elements can be used like optical elements for applying optical energy to the heart. For example, the single-point NaviStar catheter with CARTO-localization technology or any traditional single-point ablation catheter used in conjunction with St Jude's EnSite Localization system could be used.

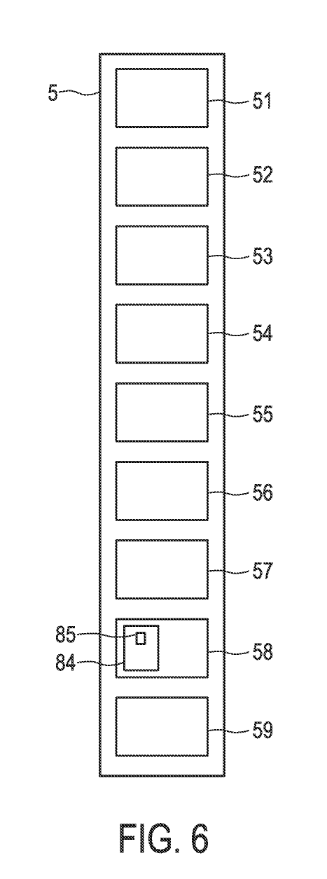

The control unit 5 comprises several further units, which are exemplarily and schematically shown in FIG. 6.

The control unit 5 comprises an electrical signal detection unit 51, which is connected via lines 30 with the electrodes 17 in order to measure an electrical signal. The lines, which connect the electrical signal detection unit 51 with the electrodes 17, are preferentially wires. The control unit 5 further comprises an electrical energy application unit 52, which is, in this embodiment, also connected to the electrodes 17 via the lines 30 in order to allow the electrodes 17 to apply electrical energy to the heart 2. Thus, in this embodiment, the electrodes 17 are able to detect electrical signals and to apply electrical energy.

The control unit 5 also comprises a temperature detection unit 53 for detecting the temperature sensed by the temperature sensors 18, which are connected with the temperature detection unit 53 via electrical conductors, in particular, via wires. If, in an embodiment, the temperature sensors are not present, the control unit 5 preferentially does not comprise the temperature detection unit 53.

An optical energy application unit 54 is connected to the energy emission elements 19 for applying optical energy to the heart 2. Preferentially, the optical energy application unit 54 is connected to the energy emission elements 19 via optical fibers. If, in an embodiment, energy emission elements 19 are not present, the control unit 5 does preferentially not comprise the optical application unit 54, which includes preferentially a laser. The optical energy application unit 54 and the energy emission elements 19 and possibly also the electrodes 17, if there are applying electrical energy, and the electrical energy application unit 52 can be adapted for performing an ablation procedure, in particular, in a heart chamber.

The control unit 5 further comprises a registration unit 55 for registering the electrodes 17 and a model of the heart 2 by using an image generated by the heart image providing unit 12 in order to indicate at which locations on the heart the electrical signals have been determined. The assignment of the electrical signals to the respective locations on the model of the heart 2 forms an electroanatomical map.

The registration by the registration unit 55 is preferentially performed by using makers 20 which are visible in an image provided by the heart image providing unit 12. In this embodiment, the markers 20 are located at the distal tip of the holding structure 50 and at the opposite and of the holding structure 50, which is adjacent to the catheter 6.

In another embodiment, in addition to or instead of the markers 20, the electrodes 17 and/or the holding structure 50 can be used as markers, if they are visible in an image of a heart image providing unit 12.

The registration unit 55 is preferentially adapted to calculate the position of each electrode 17 according to a coordinate system of the heart chamber being registered by using an image of the heart image providing unit 12. In an embodiment, the heart image providing unit is a three- or four-dimensional imaging modality, i.e. a modality generating a three- or four-dimensional image, and the registration is based on these three- or four dimensional images. If in an embodiment, the heart image providing unit provides two-dimensional images, in particular, two-dimensional fluoroscopy images, the registration unit 55 is preferentially adapted to register the electrodes 17 and the model of the heart 2 using a 2D-3D registration method in order to find the locations of the electrodes, which are shown in the two-dimensional image, on the three- or four-dimensional model.

The control unit 5 further comprises a property type determination unit 56 for determining a property type of the heart depending on at least one of a) the electroanatomical map and b) the heart image provided by the heart image providing unit. The property types, which can be determined by the property type determination unit 56, are in this embodiment complex fractionated atrial electrograms, ectopic foci, rotors, high-frequency electrograms, re-entrant circuits and slow conductions, wherein for determining these property types the electroanatomical map is used. The property type determination unit can further be adapted to determine a ganglionated plexus, scar tissue, the pulmonary vein ostium and the mitral valve annulus as property type, in particular, by using a heart image being preferentially a magnetic resonance or an X-ray computed tomography image. Moreover, the electrical signal detection unit 51, the electrical energy application unit 52 and the electrodes 17 can be adapted to measure changes in electrograms following local stimulation, wherein the property type determination unit can also be adapted to determine a ganglionated plexus and/or scar tissue and/or a re-entrant circuit as property types based on measured changes in the electrograms following the local stimulations. Furthermore, the electrodes 17, the electrical signal detection unit 51 and the electrical energy application unit 52 can be adapted to perform an entrainment mapping, wherein the property type determination unit can be adapted to determine a re-entrant circuit as property type based on the entrainment mapping.

In general, the property type determination unit 56 is adapted to determine at least one of an anatomical property type and an electrical property type of the heart 2, wherein theses property types are preferentially the above already mentioned complex fractionated atrial electrograms, ganglionated plexi, re-entrant circuits, scar tissue, rotors, pulmonary vain ostia, slow conductions and fibrosis. Furthermore, the property type determination unit 56 can be adapted to determine an ectopic focus or a mitral valve annulus as property type of the heart 2.

Since the property types have been determined based on the electroanatomical map and/or the heart image provided by the heart image providing unit, the determined properties can be assigned to locations of the heart. The control unit 5 further comprises a first site determination unit 57 for determining a first site of the heart 2, wherein the first site comprises a first property type of the determined property types. For example, the first site determination unit 57 can be adapted to determine all first sites of the heart 2, which comprise a complex fractionated atrial electrogram as a first property type. The first site determination unit 57 can comprise a selection unit for allowing a user to select a property type among the determined property types as first property type, wherein the first site determination unit 57 is adapted to determine a site comprising the selected first property type as first site.

The control unit 5 further comprises a second site determination unit 58 for determining a second site of the heart 2, wherein the second site comprises a second property type of the determined property types and wherein the second site has a causal relation to the first site. The second site determination unit 58 comprises a causality determination unit 84 for determining among the provided property types of the heart 2 a property type that has a causal relation to the first property type, wherein this determined property type is the second property type and wherein the second site determination unit 58 is adapted to determine the second site as the site where the determined second property type is located. Thus, the causality determination unit 84 determines a property type being the second property type, which is causally related to the first property type.

The causality determination unit 84 comprises a storing unit 85 for storing causal property type groups, wherein property types of a causal property type group comprise a causal relation and wherein the causality determination unit 84 is adapted to determine that the first property type and a further property type among the provided property types are causally related, if the first property type and the further property type belong to the same causal property type group. In this embodiment, following causal property type groups are stored in the storing unit 85:
complex fractionated atrial electrogram and ganglionated plexus,
re-entrant circuit and scar tissue,
rotor and pulmonary vein ostium,
ectopic focus and pulmonary vein ostium,
slow conduction and fibrosis,
slow conduction and ischemia.

For example, if the first property is a complex fractionated atrial electrogram and if the first site determination unit 57 has determined first sites comprising these complex fractionated atrial electrograms as first property type, the causality determination 84 determines a ganglionated plexus as the second property type and the second site determination unit 58 determines the sites of the heart, which comprise a ganglionated plexus, as the second sites.

The control unit 5 further comprises a causality level determination unit 59 for determining a level of causality between the first site and the second site. The causality level determination unit 59 is adapted to determine the level of causality based on at least one of a) the distance between the first site and the second site, b) the density of one of the first site and the second site within a predefined area around the other of the first site and the second site, and c) the location, in particular, the anatomical location, of at least one of the first site and the second site. The causality level determination unit 84 is preferentially adapted to choose one or several of these options for determining the level of causality depending on the first property type and/or the second property type. The distance is preferentially used in any of the above mentioned property types for determining the level of causality. The option b), i.e. the determination of the level of causality based on the density of one of the first site and the second site within a predefined area around the other of the first site and the second site, is preferentially used if one of the first and the second property types is a ganglionated plexus and if the other of the first and the second property types is a complex fractionated atrial electrogram. The option c) is also preferentially used, if at least one of the first and second property types is a ganglionated plexus and if the other of the first and second property types is a complex fractionated atrial electrogram.

In an embodiment, if two or more options are used for determining a level of causality, for each option a causality value is determined and the causality values determined for different options are weighted and summed up for determining an overall level of causality.

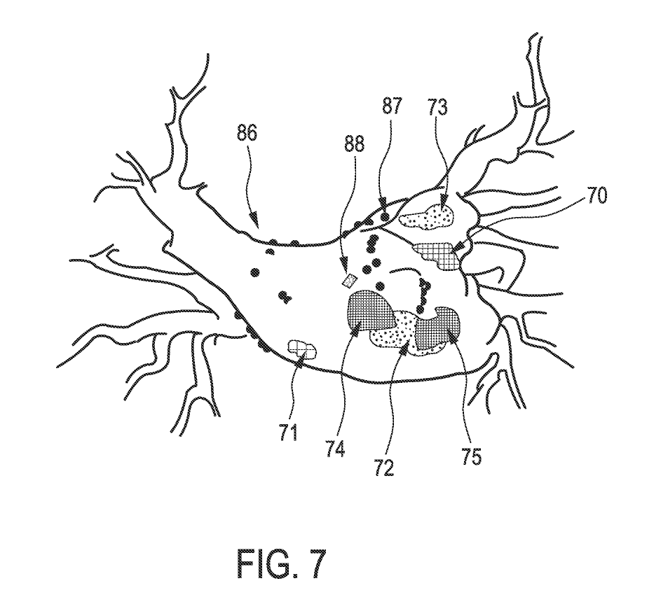

The energy application apparatus 1 further comprises a display unit 61 for displaying the first site and the second site, in particular, on the model of the heart 2 and depending on the determined level of causality. Such a displayed model 86 of the heart 2 with first sites 70, 71, 74, 75 and second sites 72, 73 is schematically and exemplarily shown in FIG. 7.

In FIG. 7, the first sites 70, 71, 74, 75 comprise as the first property type a complex fractionated atrial electrogram. The second sites 72, 73 comprise a ganglionated plexus as second property type. In this embodiment, the first sites and the second sites are shown with different colors and the brightness of the colors depends on the level of causality.

For example, the distance of the second site 72 to the first sites 74, 75 is smaller than the distance of the second site 72 to the first sites 70, 71. Furthermore, the distance of the second site 72 to the first site 71 is smaller than the distance of the second site 72 to the first site 70. Thus, if in this example the second site 72 has been selected for determining the level of causality, the level of causality is smaller for the first sites 71, 70 in comparison to the level of causality of the first sites 74, 75, and the level of causality of the first site 71 is smaller than the level of causality of the first site 70, with respect to the selected second site 72. The circles 87 indicate ablation legions.

In FIG. 7, different colors are indicated by different kinds of hatching, wherein a denser hatching indicates a higher brightness.

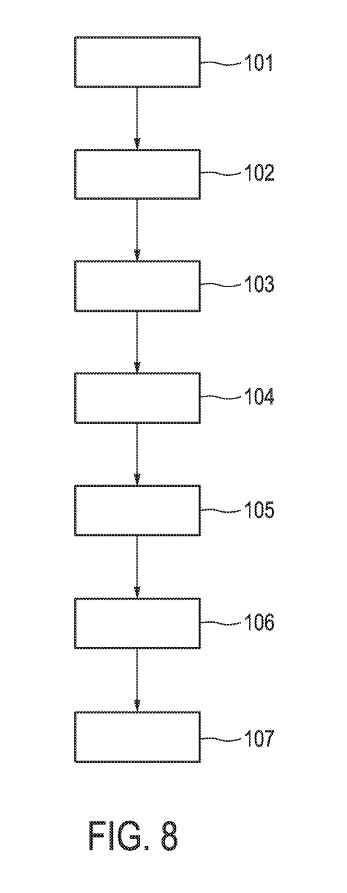

The catheter 6, the arrangement 7 of electrodes 17, the steering unit 62, the heart image providing unit 12, the electrical signal detection unit 51 and the registration unit 55 can be regarded as an electroanatomical map providing unit. This electroanatomical map providing unit, the property type determination unit 56 and optionally a further imaging modality like an x-ray computed tomography modality and/or a magnetic resonance modality constitute preferentially a property type providing unit. This property type providing unit, the first site determination unit 57, the second site determination unit 58, the causality level determination unit 59 and the display unit 61 form an embodiment of an imaging apparatus for imaging a heart in accordance with the invention. This imaging apparatus is included in the energy application apparatus 1, but this imaging apparatus could also be used without the further components or with other components for applying energy to the heart. In the following an imaging method, which uses this imaging apparatus, will exemplarily be described with reference to a flowchart shown in FIG. 8.

The arrangement 7 of electrodes 17 has been introduced into the heart 2 using the catheter 6, while the holding structure 50 is in the folded condition. In step 101, the holding structure is changed to an unfolded condition and the electrodes 17 preferentially contact the heart tissue. If in another embodiment, another kind of electrode arrangement and/or catheter is used, which does not comprise a holding structure being changeable between a folded and an unfolded condition, the step of changing a holding structure from a folded to an unfolded condition can be omitted. Furthermore, if electrical signals are measured as far-field electrical signals, the electrodes do not contact the heart tissue. The electrical signals are measured in step 102.

The heart image providing unit 12 generates at least one image of the heart 2 also showing the electrodes 17 and this image is used by the registration unit 55 for registering a model 86 of the heart 2 with the electrodes 17 within the heart 2 in step 103. Since after registration it is known at which locations of the heart the electrical signals have been acquired, an electroanatomical map is generated.

In step 104, the property type determination unit 56 determines property types of the heart at different locations of the heart based on the generated electroanatomical map and/or the image of the heart provided by the heart image providing unit 12 or provided by another imaging modality. In this embodiment, the property type determination unit determines a slow conduction, a complex fractionated atrial electrogram and a ganglionated plexus as property types.

In step 105, the first site determination unit 57 determines a first site of the heart 2, wherein the first site comprises a first property type of the provided property types, and the second site determination unit 58 determines a second site of the heart 2, wherein the second site comprises a second property type of the provided property types and wherein these determinations of the first site and the second site are performed such that the first site and the second site are causally related. In this embodiment, a complex fractionated atrial electrogram is determined as first property type of a first site and the causality determination unit 84 of the second site determination unit 58 looks in the storing unit 85 for a causal property type group, which comprises the first property type, i.e. complex fractionated atrial electrograms, and a further property type among the property types determined in step 104. In the storing unit 85 the causal property type group “complex fractionated atrial electrogram and ganglionated plexus” is stored. Therefore, the causality determination unit 84 determines the property type “ganglionated plexus” as the second property type and the second site determination unit 58 determines the locations comprising this second property type as the second sites. In this embodiment, the first sites 70, 71, 74, 75 and the second sites 72, 73 shown in FIG. 7 are determined.

The first site determination unit 57 can be adapted to determine the first site of the heart as being a first site comprising a predefined property type of the provided property types. In an embodiment, the first site determination unit 57 comprises a selection unit allowing a user to select a first property type among the provided property types, wherein the first site determination unit 57 determines the first site as the site comprising the selected first property site.

In step 106, the level of causality between the first sites and the second sites is determined. In this embodiment, the level of causality is based on the distance between the respective first site and a selected second site, i.e. for each first site a level of causality is determined, wherein if the distance is smaller the level of causality is larger. In an embodiment, the user is allowed to select a second site, for example, the second site 72 and then the levels of causality between the selected second site 72 and the first sites 70, 71, 74, 75 are determined. The first sites 74 and 75 have the shortest distance to the selected second site 72 and have therefore the highest level of causality. The first site 70 has a larger distance to the selected second site 72, and the first site 71 has the largest distance to the selected second site 72. Thus, the level of causality is smaller for the first sites 71, 70 in comparison to the level of causality of the first sites 74, 75, and the level of causality of the first site 71 is smaller than the level of causality of the first site 70, with respect to the selected second site 72. Of course, also another second site or a first site can be selected, wherein the level of causality of the second sites with respect to the selected first site can be determined.

In step 107 the determined first and second sites are shown on the model 86 of the heart on the display unit 61. The first and/or the second sites are displayed depending on the determined level of causality. In an embodiment, the first sites having a larger level of causality are shown with a larger intensity. For example, the first sites 74, 75, which have a closer distance to a selected second site 72 and, thus, a larger degree of causality in comparison to the levels of causality of the further first sites 70, 71, are shown with a larger intensity than the other first sites having a larger distance to the selected second site and, thus, a smaller level of causality. The different levels of causality can also be indicated by showing the respective sites with a different degree of transparence. For example, an increasing level of causality can be indicated by an increasing level of opaqueness.

A user like an electrophysiologist can now plan an ablation procedure based on the displayed first and second sites and perform the planned ablation procedure by using, for example, the electrode 17 and/or the energy emission elements 19.

The imaging apparatus preferentially provides an automatic interpretative electroanatomical map indicating the sites at which abnormal electrical activity was recorded by an electrophysiology (EP) mapping system and a high-level interpretation of the clinical relevance of each of these site's electrical activity, to automatically indicate clinically-relevant targets for ablation. The imaging apparatus analyzes and synthesizes one or more sets of electrical activity information, i.e. of electroanatomical maps, and displays the information in a concise manner. Thus, in the above described embodiments, preferentially several electroanatomical maps are provided and the property type determination unit determines the property types and their locations based on the several electroanatomical maps. The imaging apparatus can simultaneously display the current location of the ablation catheter or another intracardiac tool on the interpretative map. The imaging apparatus can preferentially automatically interpret all electrical activity and examine it for the property types (e.g. ectopic foci, complex fractionated electrogram sites etc.) that the user may specify before or during an ablation procedure. The imaging apparatus can preferentially further identify potentially clinically-relevant target sites based on whether the electrical measurements at a site are highly dissimilar compared to those in the rest of the atrial tissue. The imaging apparatus can be adapted to superpose an interpretative map showing the first and second sites on the one or more electroanatomical maps generated by a catheter mapping system like the electrogram providing unit described above with reference to FIG. 3. The imaging apparatus can further be adapted to automatically adapt the interpretation criteria for each property type during the mapping/ablation procedure as data is collected, to make the criteria more patient-specific.

The imaging apparatus preferentially provides an automatic interpretative electroanatomical map indicating the first and second sites at which the respective property types, in particular, abnormal electrical activity, was recorded; for each location, preferentially a high-level interpretation of the clinical relevance of this electrical activity is given by providing the first and second sites being causally related, to automatically indicate clinically-relevant targets for ablation. The imaging apparatus can be used in conjunction with any standard mapping-navigation system (such as CARTO, NavX of the Philips EP Navigator System) which yields anatomical and electrical data. The output of the mapping system consists of a set of three-dimensional coordinates, and the electrograms or electrical features recorded or computed at these coordinates, i.e. of the electroanatomical maps. The imaging apparatus then interprets the electrical signals in two ways for determining different property types. Firstly, the electrogram signals are individually analyzed for clinically-relevant characteristics e.g. a high degree of fractionation (indicating a fractionated electrogram, or CFAE, site), a low signal amplitude (indicating scar or non-conducting tissue), or a prolonged R-R interval in response to stimulation (indicating a location within the borders of a ganglionated plexus). Secondly, neighbouring electrograms can be compared to find clinically-important relative activation times e.g. earliest activation points, repetitively excited re-entrant circuits, zones of slow conduction, or sites of wavebreak.

The imaging apparatus will automatically search for many clinically-relevant classifications of abnormal electrical activity (‘property types’), including but not limited to CFAEs, slow conduction zones, scar tissue, earliest activation points, ganglionated plexi, re-entrant circuits, and sites of wavebreak. As new insights are made by the medical/research community into the important ablation targets for treating arrhythmias such as AF, other property types may be added to the apparatus. The imaging apparatus can be asked to display only the property types selected by the user. Alternatively, the imaging apparatus can display only a subset of the sites comprising the property types, i.e. e.g. of the first and the second sites, depending on the preferences of the user.

The imaging apparatus preferentially uses an extensive set of search criteria to analyze the electrical data for each of the property types. For instance, any electrogram with a maximum signal amplitude of less than 0.25 mV may be automatically classified as ‘scar’; alternatively, electrograms with continuous electrical activity at baseline and a cycle length of less than 120 ms may be automatically classified as ‘CFAE’. The imaging apparatus's search criteria can be added to or modified by the user before the procedure (if there are only certain property types that the cardiologist is interested in), during the procedure (if there are important insights that the cardiologist gains into the patient's condition during mapping), or after the procedure (to re-interpret the data in different ways); search criteria modification may even be done automatically by a central repository of knowledge (such as the American Heart Association), on a weekly/monthly/yearly basis as new clinical insights become available. The latter option will continually provide cardiologists with up-to-date knowledge on how to ablate the patient's specific arrhythmia most effectively. The cardiologist will also be able to manually modify the automatic clinical interpretation of a target site if (s)he disagrees with it.

The clinically-relevant sites, i.e. e.g. the first and second sites, can be displayed in a number of ways. It is important that the imaging apparatus synthesizes and displays the electrical activity information in a concise manner. This might be as a list or graph to indicate the frequency/3D coordinates of each property type. Preferentially, however, the tool will display the clinically-relevant sites comprising the property types on an anatomical map to yield an Interpretative Electroanatomical Map (IEM). An example of an IEM is shown in FIG. 7. An IEM displays the clinically-relevant sites using color-coding to denote property type (e.g. light blue indicates CFAEs, red indicates zones of slow conduction). The IEM can also display the electrical waveform recorded/computed at a site on the endocardial surface, if the cursor is moved over that site on the heart model.

In an embodiment, the IEM is superposed on the one or more non-interpretative electroanatomical maps generated by a catheter mapping system. Since the imaging apparatus uses data generated by the mapping system, the IEM and non-interpretative map will have the same coordinate systems (and can therefore be co-registered without difficulty). The cardiologist can superpose the IEM on any non-interpreted electroanatomical map, and thereby look at how the IEM target positions correspond to the ‘raw’, non-interpreted electrical data derived by the mapping system.

In an embodiment, the imaging apparatus simultaneously displays the current location 88 of the ablation catheter (or other intracardiac tool) on the IEM (see for example FIG. 7). Since the IEM is generated from mapping system data that is preferentially collected at a catheter tip, the catheter location and the interpretative map have the same coordinate systems (and can therefore be co-registered without difficulty).

In a further embodiment, the imaging apparatus identifies ablation targets based on the difference of the electrical measurements at that site relative to the rest of the atrial tissue. That is, the imaging apparatus does not provide the highest-level of clinical interpretation (that yields the specific property types) but instead finds locations that are potential targets by looking for electrical behavior substantially different from that of the rest of the atrium; the cardiologist can then examine the electrical behavior at these sites him/herself and decide whether to pursue them as ablation targets. A ‘difference’ of electrical behavior that might indicate an electrical abnormality could be chaotic vs. organized activity, slow vs. normal conduction velocity, circular vs. linear electrical wavefront movement, etc.

In a further embodiment, the imaging apparatus automatically and continually adapts the criteria for each property type as data is collected during an ablation procedure, to make the criteria progressively more patient-specific. Criteria adaptation is especially useful for measures of electrical behavior (such as speed of conduction) that are dependent on the patient's age, anti-arrhythmic medication and other not-necessarily disease-causing factors. It is conceivable that in an 89 year-old AF patient, the range of atrial conduction velocities is completely different from that in a 30 year-old AF patient. Therefore, it would be more appropriate to identify the patient-specific sites that exhibit outlier behavior, instead of utilizing a simple population-wide threshold value. To adapt the criteria for greater patient-specificity, the imaging apparatus will look at the distribution of the electrical behavior across the cardiac chamber, and analyze this distribution for outliers. Depending on the distribution type, this could be done by generating a histogram of the data, and looking for data points that fall more than 1.5 times the interquartile range above the third quartile or below the first quartile.

The imaging apparatus will preferentially also look at non-electrogram patient data to understand what abnormal electrical features are most important in this patient's case. For instance, an electrocardiogram (ECG) signal can be examined by the imaging apparatus in real-time to determine the instantaneous dominant abnormal electrical activity, and preferentially highlight the relevant site(s) on the IEM. If the dominant arrhythmia is premature excitation, the tool will highlight ectopic foci sites on the IEM; if flutter is indicated on the ECG, the tool will highlight sites of re-entrant electrical activity; if fibrillation, it will highlight zones of slow conduction, wavebreak and CFAEs. This feature of the imaging apparatus is especially useful in ‘stepwise’ ablation procedures, in which different arrhythmic sources are encountered in turn as the dominant sources are progressively ablated and the arrhythmia is progressively organized. The locations of the dominant sources will preferentially be highlighted by a flashing pointer or will be provided in a display read-out that indicates which property type should be focused on at this stage in the ablation procedure.

In an embodiment, maps for determining the property types can be determined by acquiring electrical data acquired from the cardiac chamber using catheter mapping technology (e.g. CARTO, NavX, the above described electrogram providing unit et cetera). Ischronal and/or isopotential maps are generated, indicating activation times and instantaneous activation patterns across the chamber, respectively. A re-entrant circuit can be identified on an isochronal map by finding a location on the map at which early activation ‘meets’ late activation with the time period of one cardiac cycle. Additionally, an isochronal map can be used to see the speed of activation of the cardiac tissue; slow activation areas can be pro-arrhythmic. Isopotential maps are excellent for detecting and localizing ectopic foci or unusual activation patterns. Fractionation maps may also be produced by the mapping system, indicating degree of fractionation of the local measured electrograms. Lastly, a voltage map reflecting the maximum electrogram amplitude (measured after local stimulation) may be generated to locate areas of scar/ischemic tissue. These maps can be regarded as low-level maps which can be used by the property type determination unit for determining the property types of the heart, for example, as follows:

Fractionation map: the degree of signal fractionation will be quantified (several algorithms already do this) and a threshold value will be set, above which an electrogram will be classed as a fractionated electrogram.

Isochronal map: Due to the complexity of the ischronal map, a re-entrant circuit can sometimes be missed or wrongly identified simply by looking at the map. In the present case, spatial feature extraction algorithms can be used to find locations that match the spatial and timing features of a re-entrant circuit.

Isopotential map: this provides timing data that is more detailed than the isochronal map but is also overwhelming in its quantity (there are as many as 100 instantaneous maps generated over a single cardiac cycle). By using spatial feature extraction, we can precisely and in real-time find locations in the cardiac chamber whose electrical activation differs in timing from its surrounding tissue.

Voltage map: we set a threshold value for the voltage amplitude, below which threshold the tissue is identified as scar.

Pacing and entrainment mapping data: the distance of the re-entrant circuit relative to a pacing or entrainment mapping catheter location can be derived by analyzing timing data. By comparing the timing data against the approximate speed of activation of the tissue (either a generic speed for cardiac tissue, or a speed estimated from the isopotential/isochronal maps) an area in which the re-entrant circuit pathway is likely to be located can be specified. This is useful for an electrophysiologist as he/she attempts to move the catheter towards the pathway for ablation.

ECG data: the chamber octant containing the ectopic focus can be automatically estimated from the morphologies of the P or Q wave in the 12-lead chest ECG.

A first property type and a second property type among the determined property types are selected such that they are causally related, corresponding first and second sites are determined, which comprise the first property type and the second property type, respectively, and the first and second sites are displayed on the display unit 61. The cardiologist can now identify synergies between these risk areas, i.e. between the first and second sites. This is of value because the importance of ablating a risk area is increased if there are additional indications that the area is important for the maintenance of the arrhythmia e.g. if the region is close to scar tissue, and has also been interpreted as a re-entry circuit, it is more likely to be a focus of ablation.

If a user has selected at least one of the first and second sites, the selected site is preferentially ablated using an ablation catheter, for example, the electrodes 17 or the energy emitting elements 19. Preferentially, also the locations of ablation lesions are shown by the display unit 61.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or devices may fulfill the functions of several items recited in the claims. The mere fact that certain measures are recited in mutually different dependent claims does not indicate that a combination of these measures cannot be used to advantage.

Calculations and determinations, like the registration or the determination of property types and first and second sites, performed by one or several units or devices can be performed by any other number of units or devices. The calculations and determinations and/or the control of the imaging apparatus in accordance with the imaging method can be implemented as program code means of a computer program and/or as dedicated hardware.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

1. An imaging apparatus for imaging a heart, wherein the imaging apparatus comprises:
a property type providing unit (56; 91) for providing property types of the heart (2) at different locations of the heart (2),
a first site determination unit (57; 92) for determining a first site (70, 71, 74, 75) of the heart (2), wherein the first site (70, 71, 74, 75) comprises a first property type of the provided property types,
a second site determination unit (58; 92) for determining a second site (72, 73) of the heart (2), wherein the second site (72, 73) comprises a second property type of the provided property types and wherein the second site (72, 73) has a causal relation to the first site (70, 71, 74, 75),
a display unit (61) for displaying the first site (70, 71, 74, 75) and the second site (72, 73).

2. The imaging apparatus as claimed in claim 1, wherein the property type providing unit (56; 91) is adapted to provide at least one of an anatomical property type and an electrical property type of the heart (2).

3. The imaging apparatus as claimed in claim 1, wherein the property type providing unit (56; 91) is adapted to provide at least one of a complex fractionated atrial electrogram, a ganglionated plexus, a re-entrant circuit, scar tissue, a rotor, a pulmonary vein ostium, a slow conduction and fibrosis as a property type of the heart.

4. The imaging apparatus as claimed in claim 1, wherein the second site determination unit (58; 92) comprises a causality determination unit (84; 96) for determining among the provided property types of the heart (2) a property type that has a causal relation to the first property type, wherein this determined property type is the second property type and wherein the second site determination unit (58; 92) is adapted to determine the second site (72, 73) as the site where the determined second property type is located.

5. The imaging apparatus as claimed in claim 4, wherein the causality determination unit (84; 96) comprises a storing unit (85; 97) for storing causal property type groups, wherein property types of a causal property type group comprise a causal relation and wherein the causality determination unit (84; 96) is adapted to determine that the first property type and a further property type among the provided property types are causally related, if the first property type and the further property type belong to the same causal property type group.

6. The imaging apparatus as claimed in claim 5, wherein at least one of the following causal property type groups is stored in the storing unit (85; 97):
complex fractionated atrial electrogram and ganglionated plexus,
re-entrant circuit and scar tissue,
rotor and pulmonary vein ostium,
ectopic focus and pulmonary vein ostium,
slow conduction and fibrosis,
slow conduction and ischemia.

7. The imaging apparatus as claimed in claim 1, wherein the imaging apparatus further comprises a causality level determination unit (59; 98) for determining a level of causality between the first site (70, 71, 74, 75) and the second site (72, 73).

8. The imaging apparatus as claimed in claim 7, wherein the causality level determination unit (59; 98) is adapted to determine the level of causality based on the distance between the first site (70, 71, 74, 75) and the second site (72, 73).

9. The imaging apparatus as claimed in claim 7, wherein the causality level determination unit (59; 98) is adapted to determine the level of causality based on the density of one of the first site (70, 71, 74, 75) and the second site (72, 73) within a predefined area around the other of the first site (70, 71, 74, 75) and the second site (72, 73).

10. The imaging apparatus as claimed in claim 7, wherein the causality level determination unit (59; 98) is adapted to determine the level of causality based on the location of at least one of the first site (70, 71, 74, 75) and the second site (72, 73).

11. The imaging apparatus as claimed in claim 7, wherein the display unit (61) is adapted to display the first site (70, 71, 74, 75) and/or the second site (72, 73) depending on the determined level of causality.

12. An energy application apparatus for applying energy to a heart, wherein the energy application apparatus comprises an energy application unit for applying energy to the heart and an imaging apparatus as defined in claim 1.

13. An imaging method for imaging a heart, wherein the imaging method comprises following steps:
providing property types of the heart (2) at different locations of the heart (2),
determining a first site (70, 71, 74, 75) of the heart (2), wherein the first site (70, 71, 74, 75) comprises a first property type of the provided property types,
determining a second site (72, 73) of the heart (2), wherein the second site (72, 73) comprises a second property type of the provided property types and wherein the second site (72, 73) has a causal relation to the first site (70, 71, 74, 75),
displaying the first site (70, 71, 74, 75) and the second site (72, 73).

14. An imaging computer program for imaging a heart, the computer program comprising program code means for causing an imaging apparatus as defined in claim 1 to carry out the steps of the imaging method as defined in claim 13, when the computer program is run on a computer controlling the imaging apparatus.
